(19) United States
(12) Patent Application Publication
(10) Pub. No.: US 2006/0241377 A1
(43) Pub. Date: Oct. 26, 2006

(54) System and method for bone strength assessment

(76) Inventor: Timothy W. James, Goleta, CA (US)

Correspondence Address: NEIL K. NYDEGGER;NYDEGGER & ASSOCIATES, 348 Olive Street, San Diego, CA, 92103, US

(21) Appl. No.: 11/074,581

(22) Filed: Mar. 8, 2005

(51) Int. Cl. A61B 5/05

(52) U.S. Cl. 600/410

(57) ABSTRACT

A device for non-invasively assessing bone strength includes an encoder for establishing a spatial frequency encode that is in a k-space vector form (λₖ=mi+nj). Importantly, the encode has a magnitude that corresponds to a spatial characteristic that is indicative of bone strength. A magnet creates a magnetic field, and an antenna is used to radiate the bone in the magnetic field with a single encoded energy pulse to generate an encoded response signal from the bone. A computer/comparator then compares the encoded response signal with a base value to assess bone strength.

### FIELD OF THE INVENTION

The present invention pertains generally to medical diagnostic devices. More particularly, the present invention pertains to NMR techniques that are useful for non-invasively obtaining information about the internal tissues of humans and animals. The present invention is particularly, but not exclusively, useful for obtaining information about bone tissue that can be diagnostically used to assess the strength of the bone.

### BACKGROUND OF THE INVENTION

All bones of the body are similar to each other in that they are all composed of substantially the same types of structures. More specifically, the composition of bones is seen to be of essentially two kinds of bony tissue. These are: cortical and cancellous bone. In particular, the more dense cortical bone is usually the exterior of most bones, while the cancellous tissue is usually internal. Unlike cortical bone, cancellous bone is made up of an open lattice work of intersecting osseous plates and bars (trabeculae) that are lined with bone forming and bone resorbing cells. The remaining spaces are filled with marrow. Together, the trabecula join to form a reticular structure that resembles a lattice-work and is a significant contributor to the overall strength of a bone.

Cortical bone is much more dense in its structure than is cancellous bone. Accordingly, the less dense and weaker cancellous bone has greater blood flow and metabolic activity than does cortical bone. As a result, it is more susceptible to metabolic changes in the body such as disease, hormone imbalance, etc. Osteoporosis is therefore reflected in the cancellous bone much earlier than in cortical bone. The relative quantity of these two kinds of bone (i.e. cortical and cancellous) varies in different bones, and will also vary in different parts of the same bone, as strength or lightness is requisite.

In their respective structures, the difference between cortical and cancellous bone depends upon the different amounts of solid matter in the respective tissues and, supplementarily, on the size and number of marrow spaces between the solid matter. For cortical tissue, the marrow spaces (i.e. non-osseous and non-structural regions in the tissue) are smaller, and the osseous solid matter surrounding them is more abundant. On the other hand, in cancellous tissue the spaces are large and the solid matter is relatively smaller in quantity. As implied above, the trabecular structure of cancellous bone can be likened to an assembly of mechanical trusses. The purpose of this structure is two-fold. For one, it provides mechanical support and metabolic activity for the overlying cortical bone. For another, it also reduces weight. Consequently, for all bones, and in particular weight bearing bones, the continued strength of the bone will be dependent on the continued metabolic balance between bone resorbing and bone forming cells. In this regard, the integrity and density of the cancellous tissue is a sensitive indicator of this balance and a significant contributor to overall bone strength.

As is well known, a loss of bone strength can have many adverse consequences. For example, osteoporosis, which is a pathologic metabolic condition causing thinning and weakness of bones, may be accompanied by pain, deformities, and pathological fractures. Further, and perhaps more importantly, osteoporosis is insidious in its development and is often only seen clinically, after irreversible bone loss. In this case, for example, bone loss can result in disabling and life threatening fractures of the spinal column or hips. There are numerous causes for osteoporosis including disease states, metabolic abnormalities, and physical disuse. In any event, early awareness of any loss of bone strength is critical to diagnosing and successfully treating osteoporosis. One well-known, non-invasive technique for evaluating internal tissue involves nuclear magnetic resonance (NMR) techniques.

In the context of nuclear magnetic resonance (NMR), it is known that different tissues in the body have different signal characteristics. Based on this phenomenon, NMR techniques have been employed to non-invasively obtain information about internal tissue inside the body (e.g. MRI). More specifically, this information is obtained by evaluating signal responses from the various tissues that are of interest. Although this has been typically accomplished by imaging tissue using so-called homogeneous magnetic fields, it is now known that non-homogeneous magnetic fields can also be effectively used for these purposes.

By way of background, frequency (f) is the rate of repetition of a periodic disturbance. On the other hand, wavelength (λ) is defined as the distance, measured radially from a source between two successive points in free space, at which an electromagnetic, acoustic, or other waveform has the same phase. In this context, phase is a fraction of a cycle of a periodic waveform (usually sinusoidal) which has been completed at a specific reference time, e.g. at the start of a cycle of a second waveform of the same frequency. In general use, phase is expressed as an angle, with one complete cycle corresponding to 2π radians or 360°. For an electromagnetic wave (e.g. an r.f. wave), wavelength (λ) and frequency (f) are related to each other by the expression λ=c/f where “c” is the velocity of light.

Phase and frequency encoding are well-known NMR techniques that are often used for distinguishing signals when they are used to image (i.e. MRI) or identify different characteristics of internal tissues.

In k-space each spatial frequency has a “k” number (i.e. a wave number) which is indicative of its spatial frequency/wavelength. More particularly, in two dimensions, the “k” number is of the form mi+nj wherein “i” and “j” are used to define direction, and “m” and “n” are used to define magnitude. Taken together, mi+nj is a K-space identifier that defines a vector (λₖ) which has both a magnitude and a direction. As will be appreciated by the skilled artisan, a three dimensional vector can be defined for similar purposes in k-space. In this case, the “k” number is of the form mi+nj+lk. When used in NMR or MRI, a plethora of phase and/or frequency encodes are used across k-space to obtain a different spatial frequency based signal for each encode. For MRI, these signals are then transformed (e.g. Fourier Transform) into a signal that can be used to generate pixel values in an image of the tissue being imaged. It happens, however, that even before the spatial frequency based signals are transformed, they include useable information about the tissue being imaged.

Insofar as bone tissue is concerned, calcified hard bone has a significantly different NMR response signal than does the bone marrow. With this in mind, the interaction between the radiation and the bone involves both an excitation by the electromagnetic wave and the subsequent emission of a response signal. Importantly, a response will depend on the “k” number for the wavelength of the encoded spatial wavelength (λₖ). For instance, where the spatial wavelength in k-space (i.e. the magnitude of λₖ) is either much greater, or much less than the distance “s” across spaces in the tissue (i.e. λₖ>>s, or λₖ<<s) a relatively weak response can be expected. On the other hand, when the wavelength and the distance across spaces in the tissue are nearly equal (i.e. λₖ≈s), a relatively strong response can be expected. A consequence of this in the case of bone tissue, is that the resultant responses will be different depending on whether solid matter (i.e. reticular structure) is encountered. Based on these responses, the characteristic dimension (spatial wavelength) of spaces in the reticular structure of cancellous tissue (i.e. bone density) can then be assessed.

Due to the anisotropic nature of cancellous bone tissue, it is desirable to evaluate the tissue from several different perspectives (e.g. directions). Fortunately, these different perspectives can be implemented in the two dimension spatial frequency domain of k-space merely by changing the values of “m” and “n” in the k-space identifier (mi+nj). In the more general case of three dimensions, the values of “m,” “n” and “l” can be changed. Further, for specific applications wherein a selected magnitude for λₖ is of particular interest, different perspectives can be realized while the magnitude of the k number remains substantially constant. For example, in two dimensional k-space, the following k-space vectors (e.g. λₖ) all have different orientations (i.e. perspectives) but they have substantially the same magnitudes: 10i+0j; 7i+7j; and 0i+10j.

With the above in mind, it is well known that under certain conditions a bone can create a measurable response signal when placed in a magnetic field and radiated with pulses of energy. Accordingly, radiation can be created that corresponds to those conditions, and the response to this radiation can then be used to determine whether, in fact, those conditions are present.

Heretofore, NMR techniques for imaging bone tissue have employed a three dimensional matrix of spatial frequencies. A 3D Fourier transform of this matrix then provides all of the information that is required to generate a three dimensional image of the bone tissue. There is, however, sufficient information in an appropriate subset of the entire three-dimensional frequency matrix (K-space matrix) for the purpose of assessing bone strength. Moreover, this information is retrievable from the initial NMR encoded spatial frequency responses, without any further need for employing Fourier transform methodology. Specifically, encodings having selected “K” numbers (λₖ=mi+nj), as indicated above, can be an appropriate subset.

In light of the above, it is an object of the present invention to provide a device for non-invasively assessing bone strength, in vivo, based on quantitative numerical data obtained using NMR techniques. Another object of the present invention is to provide a device for assessing bone strength using predetermined energy pulses that are encoded with selected k-space identifiers (spatial frequency domain) to obtain frequency responses that are indicative of structural changes (spatial domain) in the bone that is being assessed. Still another object of the present invention is to provide a device for assessing bone strength, in vivo, within a relatively short period of time, and without unduly confining or restraining the patient during the assessment. Yet another object of the present invention is to provide a device for assessing bone strength that is relatively simple to manufacture, is easy to use, and is comparatively cost effective.

### SUMMARY OF THE INVENTION

In accordance with the present invention, a system for non-invasively assessing bone strength includes an antenna for generating pulses of energy (e.g. r.f. energy), and an encoder for encoding these individual pulses of the energy. For the present invention, this encoding is accomplished using a spatial frequency encode (λₖ) that is selected for each individual pulse according to a k-space identifier. In detail, for a two dimensional analysis, the k-space identifier can be expressed in a vector form (λₖ=mi+nj). For a three dimensional analysis, a k-space identifier in the form (λₖ=mi+nj+e k) may be used. In either case, it has a magnitude and a direction in k-space. For purposes of the present invention, however, sufficient accuracy in assessing bone strength is achievable using the two dimensional form of the k-space identifier (λₖ=mi+nj). In this case, it is necessary to select slices from the sample so that meaningful data from encoded response signals can be received.

The system of the present invention also includes a magnet for creating a magnetic field in an open region. This magnetic field may be either homogeneous or inhomogeneous and, in either case, the open region is dimensioned so that the bone being assessed may be properly positioned in the magnetic field for strength assessment. For practical reasons, the calcaneus (heel) bone of a patient's foot is the most convenient and likely bone to be selected for this assessment. Another possible candidate bone is the distal radius.

In the operation of the system of the present invention, a patient is required to first position his/her foot in the open region. The antenna is then activated to radiate the bone in the magnetic field with an excitation (i.e. an energy pulse or series of pulses). Importantly, as mentioned above, these radiation pulses (excitations) are followed with a spatial frequency encode. The consequence here is that the excited bone will generate encoded response signals. The response signals are then received by a computer and compared with a base value to assess bone strength.

Several aspects of the present invention are of particular importance. First, it is to be appreciated that it is theoretically possible to assess bone strength with the present invention, using only a single encoded pulse. Due to the anisotropic nature of bone tissue, however, more than a single pulse may be needed. If so, the responses from several pulses, each taken from a different perspective, may be required. Second, the magnitude of the encode, which is established in the frequency domain of k-space, is selected to correspond to a dimension, in the spatial domain, that is characteristic of the bone being assessed. For purposes of assessing bone strength, the dimension of interest in the spatial domain is the distance “s” (i.e. the distance across spaces in the bone tissue). Accordingly, the wavelength of the phase encode (λₖ) preferably corresponds to a spatial domain in a range of 0.1 to 2 mm. In many instances, a distance of around one half millimeter (λₖ=0.5 mm) is suitable.

In a typical operation, rather than involving the time and expense of radiating bone tissue with a plethora of pulses, having a range of different encoded wavelengths to generate an image, the present invention recognizes that pulses having essentially a single or smaller range of encoded wavelength(s) will suffice. As indicated above, the selected wavelength for this purpose preferably corresponds with the anatomical aspects presented by healthy bone (e.g. λₖ≈0.5 mm). The encoder will then establish a plurality of phase encodes for sequentially encoding a succession of individual pulses (e.g. five pulses). The bone to be assessed is then radiated with these encoded pulses to create encoded response signals.

As appreciated by the present invention, the encoded response signals are essentially quantified numerical values. Consequently, bone strength assessment in accordance with the present invention is accomplished by comparing the resultant numerical values of response signals with an empirically obtained numerical value that is indicative of healthy bone (i.e. a base value). Thus, according to whether, and how, the encoded response signals differ from the base value, an assessment of bone strength can be made.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of the system of the present invention;

FIG. 2A is a cross section of normal, healthy tissue in the calcaneus bone as seen along the line 2-2 in FIG. 1;

FIG. 2B is a cross section of osteoporotic tissue in the calcaneus bone as seen along the line 2-2 in FIG. 1;

FIG. 3 is a graphical plot of two dimensions of k-space as seen along the line 2-2 in FIG. 1; and

FIG. 4 is a bar graph showing responses from normal and osteoporotic bone tissue as a function of the signal perspective with constant spatial encodes (k-space identifier) used by the invention of the present invention.

### DESCRIPTION OF THE PREFERRED EMBODIMENTS

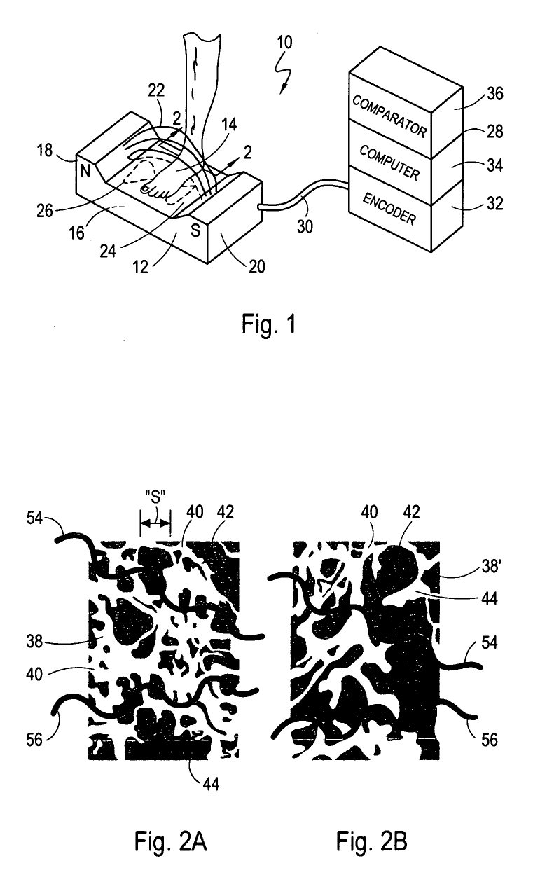

Referring initially to FIG. 1, a system (device) for assessing the bone strength of a patient in accordance with the present invention is shown and is generally designated 10. As shown, the device 10 includes a platform 12 upon which the foot 14 of a patient (not shown) can be positioned for bone strength assessment. Further, the platform 12 includes a magnet 16 having a North pole 18 and a South pole 20 for generating a magnetic field 22. In particular, the North pole 18 is distanced from the South pole 20 to establish an open region 24 on the platform 12 for receiving and positioning the foot 14 in the magnetic field 22. For purposes of the present invention, the magnetic field 22 may be either a homogeneous or an inhomogeneous magnetic field. Further, the magnet 16 may be of any type magnet well known in the pertinent art, such as a permanent magnet.

Still referring to FIG. 1 it will be seen that the device 10 also includes a transmit/receive antenna 26 (shown in phantom). Specifically, the antenna 26 is mounted or otherwise positioned on the platform 12 for the purpose of radiating pulses of energy (e.g. r.f. energy) into the foot 14 while it is positioned in open region 24. As contemplated for the present invention, these pulses of energy (not shown) can be generated, and their responses received, by antenna 26 in a manner well known in the pertinent art.

FIG. 1 also shows that the platform 12 is electronically connected to a console 28 via conduit 30. As shown, the console 28 includes an encoder 32 (preferably, a gradient system encoding device), a computer 34 and a comparator 36. For purposes of the device 10, the components of the console 28 (i.e. the encoder 32, computer 34 and comparator 36) can all be of types well known in the pertinent art. The functional uses of these components for the present invention are, however, specifically implemented for assessing bone strength in the foot 14. As a practical matter, and as a matter of convenience, the device 10 uses these components for assessing the bone strength in the calcaneus bone of the foot 14.

FIG. 2A shows a cross sectional view of a healthy calcaneus bone in the foot 14. Specifically, FIG. 2A shows that cancellous tissue 38 in the interior of the calcaneus is made up of fibers 40 (i.e. solid matter such as the plates and bars (trabeculae) mentioned above), and spaces (cavities) 42 that are filled with marrow. More specifically, the fibers 40 are separated by spaces 42 to create a reticular structure 44 for the cancellous tissue 38. An anatomical characteristic of this reticular structure 44 is that, for normal, healthy cancellous tissue 38, the distance “s” across the spaces 42 will be generally around one half millimeter (s=0.5 mm). On the other hand, when comparing unhealthy (i.e. osteoporotic) tissue 38′ (FIG. 2B) with healthy tissue 38 (FIG. 2A) it will be seen that distances corresponding to the distance “s” in healthy tissue are generally greater in the unhealthy tissue 38′, and that the spaces 42 between fibers 40 are much more prevalent. In order to tell the difference between these two conditions (i.e. normal tissue 38 vs. unhealthy tissue 38′) the device 10 of the present invention uses spatial encoding of pulses of energy radiated into the foot 14 by encoder 32 (encoding device) and antenna 26.

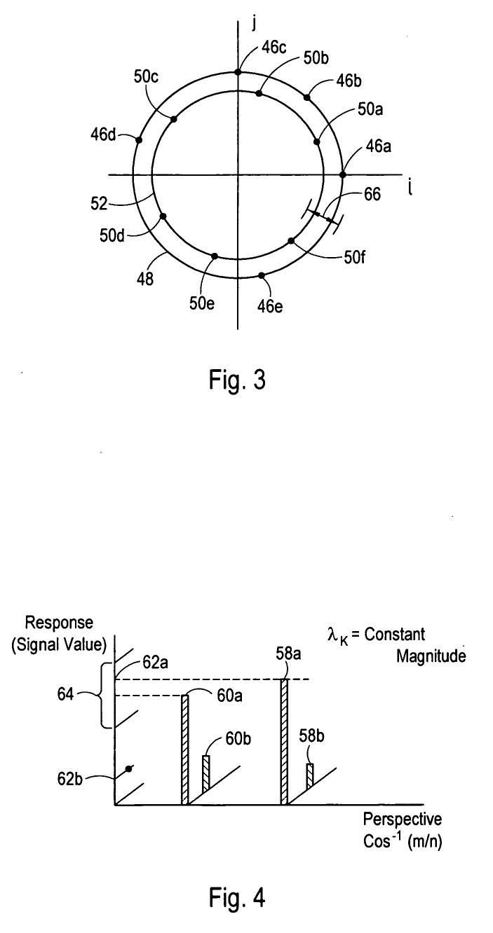

In accordance with the concepts of the present invention, the spatial encodes (λₖ) that are radiated into the foot 14 are established by the encoder 32. Specifically, the encodes (λₖ) are established in accordance with a k-space (frequency domain), such as is depicted in FIG. 3. In overview, and with reference to FIG. 3, it will be seen that each encode (λₖ) can be expressed in a vector form as λₖ=mi+nj. For example, the encode 46a is expressed in k-space as λₖ=10i+0j. In this example, the encode 46a represents a vector having a magnitude of “10” and a direction along the “i” axis. Similarly, the encode 46b is a vector expressed as λₖ=˜7i+˜7j, and the encode 46c is a vector expressed as λₖ=0i+10j. Carrying this a step further, it will be appreciated that other encodes can be identified which are located on the same magnitude circle 48 with encodes 46a-c (e.g. encodes 46d and 46e). In this case, they all have a same magnitude (e.g. “10”), but each has a different direction. For purposes of the present invention, a plurality of encodes (λₖ) that all have a same magnitude, but different directions, are defined as a succession. Thus, all encodes of a succession will lie substantially on a same magnitude sphere or in a two dimensional circle in k-space (e.g. circle 48). Using this definition, the encodes 50a-f that are shown located on the magnitude sphere or in a two dimensional circle 52 in FIG. 3 represent a different succession.

In the operation of the device 10 of the present invention, a patient places his/her foot 14 in the open region 24 of the platform 12. The antenna 26 then radiates a succession of excitation pulses into the foot 14. For purposes of this disclosure, consider a succession of energy pulses which includes an encoded pulse 54, that has been encoded with the encode 46a, and an encoded pulse 56, that has been encoded with the encode 46b. In accordance with the present invention, if the foot 14 has normal bone tissue (FIG. 2A) the encoded pulse 54 is expected to generate a response signal 58a (see FIG. 4). On the other hand, if the foot 14 is osteoporotic (FIG. 2B) a same encoded pulse 54 is expected to generate a different response signal 58b (see FIG. 4). Likewise, but from another perspective, when the foot 14 is radiated with the encoded pulse 56 (encode 46b) it will generate response signals 60a and 60b. In each instance, the response signals 58 and 60 will be indicative of the particular condition in the bone. Foot 14 has been discussed here, only by way of example. As intended for the present invention the strength (condition) of other bones may be similarly assessed.

It is an important aspect of the present invention that the response signals 58 and 60 have a measurable and quantifiable value that can be used directly to assess bone strength. Significantly, the measured values from these response signals need not be transformed in order for them to be evaluated by the computer 34 and comparator 36. For instance, if the encoded pulse 54 generates a response signal 58a, having a signal value 62a that lies within a predetermined base value range 64, the indication is there is normal tissue in the bone. On the other hand, if this same encoded pulse 54 generates a response signal 58b that has a signal value 62b that lies outside the range 64, the indication is there is osteoporotic tissue in the bone.

As appreciated by the present invention, the anisotropic nature of bone tissue effectively requires that measurements for bone strength assessment be taken from a plurality of perspectives. Accordingly, a succession of encoded pulses (e.g. five or more) will normally be used for this purpose, and employed with a view toward mutually confirming and verifying the collective response signals. Also, several successions of encoded pulses can be used to further confirm and verify an assessment of bone strength. Specifically, within the restrictions imposed by the anatomical distance “s” in the reticular structure 44, each succession of encoded pulses will typically use encodes from the same magnitude circles (e.g. circles 48 and 52) that are within a range 66 (see FIG. 3) that corresponds to normal, healthy cancellous bone tissue.

While the particular System and Method for Bone Strength Assessment as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for assessing bone strength which comprises:
a magnetic means for creating a magnetic field, said magnetic means being formed with an open region for receiving the bone therein;
a means for exciting the bone in the magnetic field with an r.f. excitation;
a means for encoding the excited bone in the magnetic field, wherein the encoding has a selected spatial frequency encode according to a k-space identifier to generate measurable encoded response signals from the bone;
a computer means for receiving each encoded response signal to generate a respective signal value therefrom, wherein the signal value is indicative of the strength of the bone; and
a comparator for comparing each signal value with a predetermined base value to assess bone strength.

2. A device as recited in claim 1 wherein the encoding means generates a succession of at least five r.f. pulses.

3. A device as recited in claim 1 wherein each k-space identifier corresponds to a substantially same wavelength (λₖ).

4. A device as recited in claim 3 wherein the magnitude of the wavelength (λₖ) corresponds to a spatial dimension of approximately one half millimeter (λₖ=0.5 mm).

5. A device as recited in claim 1 further comprising a plurality of separate successions, wherein the k-space identifiers of each succession correspond to a substantially same magnitude wavelength (λₖ), and wherein different successions have respectively different k-space identifiers corresponding to different magnitude wavelengths (λₖ₁ et seq.).

6. A device as recited in claim 1 wherein the k-space identifier is in a vector form (λₖ=mi+nj) wherein “i” and “j” relate to direction, and wherein “m” and “n” relate to a magnitude of the spatial frequency encode.

7. A device as recited in claim 1 wherein the signal value and the base value are represented as numbers.

8. A device as recited in claim 1 wherein the bone is a calcaneus bone.

9. A device as recited in claim 1 wherein the magnetic field in inhomogeneous.

10. A device for assessing bone strength which comprises:
an encoder for establishing a spatial frequency encode according to a k-space identifier, wherein the k-space identifier is in a vector form (λₖ=mi+nj) and has a magnitude selected from a predetermined range of magnitudes;
a magnet for creating a magnetic field in an open region, wherein the open region is dimensioned for receiving the bone therein;
an antenna for radiating the bone in the magnetic field with an energy pulse; and
a computer for receiving encoded response signals from the bone wherein the response signals are encoded with the spatial frequency encode, and wherein the computer compares the encoded response signal with a base value to assess bone strength.

11. A device as recited in claim 10 wherein the magnitude of the wavelength (λₖ) corresponds to a spatial dimension of approximately one half millimeter (λₖ=0.5 mm).

12. A device as recited in claim 10 wherein said encoder establishes a plurality of encodes for sequentially encoding a succession of individual pulses, wherein each pulse has a different encode, and all pulses have a substantially same magnitude.

13. A device as recited in claim 12 further comprising a plurality of separate successions, wherein the k-space identifiers of each succession correspond to a substantially same magnitude wavelength (λₖ), and wherein different successions have respectively different k-space identifiers corresponding to different magnitude wavelengths (λₖ₁ et seq.).

14. A device as recited in claim 10 wherein the encoded response signal and the base value are numbers.

15. A device as recited in claim 10 wherein the magnetic field is inhomogeneous.

16. A method for assessing bone strength which comprises the steps of:
positioning the bone in a magnetic field;
exciting the bone in the magnetic field with an excitation;
encoding the excited bone according to a k-space identifier to generate an encoded response signal therefrom, wherein the k-space identifier is in a vector form (λₖ=mi+nj) and has a magnitude selected from a predetermined range of magnitudes; and
comparing the encoded response signal with a base value to assess bone strength.

17. A method as recited in claim 16 wherein said encoding step comprises the steps of:
selecting a magnitude for λₖ from a range corresponding to a predetermined spatial dimension in an x-y plane; and
changing the k-space identifier to sequentially encode a succession of individual pulses, with each pulse having a different encode and a substantially same magnitude.

18. A method as recited in claim 17 further comprising the step of repeating said selecting step to vary the magnitude of k.

19. A method as recited in claim 16 wherein the encoded response signal and the base value are numbers.

20. A method as recited in claim 16 wherein the magnetic field is inhomogeneous.
